(19) United States
(12) Patent Application Publication
(10) Pub. No.: US 2009/0105322 A1
(43) Pub. Date: Apr. 23, 2009

(54) PHARMACEUTICAL COMPOSITION COMPRISING VALSARTAN

(76) Inventors: Jay Norman Cohn, Minneapolis, MN (US); Robert Dean Glazer, Piscataway, NJ (US); Robert Latini, Milan (IT); Aldo Pietro Maggioni, Florence (IT); Gianni Tognoni, Milan (IT)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 12/341,669

(22) Filed: Dec. 22, 2008

Related U.S. Application Data
(63) Continuation of application No. 10/556,260, filed on Feb. 3, 2006, now abandoned.
(60) Provisional application No. 60/471,137, filed on May 16, 2003.

(51) Int. Cl. A61K 31/41; A61P 9/00

(52) U.S. Cl. 514/381

(57) ABSTRACT

The invention relates to pharmaceutical compositions and a method of preventing or reducing the incidence of AF and thereby reducing the risk of morbidity and mortality in patients having symptomatic heart failure comprising administering to such patient an effective amount of valsartan, or pharmaceutically acceptable salts thereof, alone or in combination with another therapeutic agent, optionally in the presence of a pharmaceutically acceptable carrier.

This application is a continuation application of Ser. No. 10/556,260, filed Feb. 3, 2006, which is a 371 of Application Number PCT/EP04/005204, filed May 14, 2004, which claims benefit of Application No. 60/471,137, filed May 15, 2003, now abandoned.

### BACKGROUND OF THE INVENTION

Angiotensin II receptor blockers (ARBs), such as valsartan, are known as anti-hypertensive agents which selectively block the binding of angiotensin II (Ang II) to the AT₁-receptor causing vasodilatation, and diminish aldosterone secretion. ARBs are also known to treat congestive heart failure (CHF).

Atrial fibrillation (AF) in patients with heart failure is generally considered a negative prognostic factor. Recent studies indicate that the incidence of AF might be decreased by renin-angiotensin system (RAS) inhibitors. The identification of the independent predictors of AF and a treatment able to prevent its occurrence is likely to improve patients outcome. We have now discovered in large clinical studies that administration of valsartan in a cohort of patients reduces AF.

### SUMMARY OF THE INVENTION

In one embodiment, the present invention relates to a method of reducing the occurrence of AF and thereby reducing the risk of morbidity and mortality in patients having symptomatic heart failure comprising administering to such patient an effective amount of valsartan, or pharmaceutically acceptable salts thereof optionally in the presence of a pharmaceutically acceptable carrier.

In another embodiment, the present invention relates to a method of preventing or reducing the incidence of AF and thereby reducing the risk of morbidity and mortality in patients having symptomatic heart failure comprising administering to such patient an effective amount of valsartan and at least another therapeutic agent optionally in the presence of a pharmaceutically acceptable carrier.

In another embodiment, the present invention relates to a pharmaceutical composition comprising valsartan, alone or in combination with at least another therapeutic agent in the presence of a pharmaceutically acceptable carrier for the prevention or treatment of AF in patients having symptomatic heart failure.

Other objects, features, advantages and aspects of the present invention will become apparent to those of skill from the following description. It should be understood, however, that the following description and the specific examples, while indicating preferred embodiments of the invention, are given by way of illustration only. Various changes and modifications within the spirit and scope of the disclosed invention will become readily apparent to those skilled in the art from reading the following description and from reading the other parts of the present disclosure.

### DETAILED DESCRIPTION OF THE PREFERRED EMBODIMENTS

In one embodiment, the present invention relates to a method of preventing or reducing the incidence of AF and thereby reducing the risk of morbidity and mortality in patients having symptomatic heart failure comprising administering to such patient an effective amount of valsartan, or pharmaceutically acceptable salts thereof optionally in the presence of a pharmaceutically acceptable carrier.

Valsartan is the AT₁-receptor antagonist (S)-N-(1-carboxy-2-methyl-prop-1-yl)-N-pentanoyl-N-[2; (1H-tetrazol-5-yl)biphenyl-4-yl-methyl]amine of formula (I)

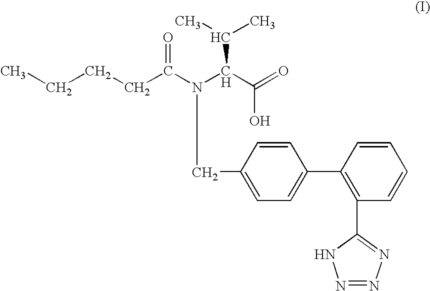

and the pharmaceutically acceptable salts thereof. Valsartan is disclosed in EP 0443983 A and U.S. Pat. No. 5,399,578, the disclosures of which are incorporated herein in their entirety as if set forth herein.

In another embodiment, the present invention relates to a method of preventing or reducing the incidence of AF and thereby reducing the risk of morbidity and mortality in patients having symptomatic heart failure comprising administering to such patient an effective amount of valsartan and at least another therapeutic agent optionally in the presence of a pharmaceutically acceptable carrier.

The term “at least one therapeutic agent” shall mean that in addition to valsartan one or more, for example two, furthermore three, active ingredients as specified according to the present invention can be combined.

The therapeutic agents which may be combined with valsartan include, but are not limited to, anti-hypertensive agents, anti-obesity agents, anti-diabetic agents, beta-blockers, inotropic agents and hypolipidemic agents.

Preferred antihypertensive therapeutic agents according to the invention are Angiotensin converting enzyme (ACE) inhibitors.

Suitable ACEIs for use in the present invention include benazepril, captopril, cilazapril, enalapril, enalaprilat, fosinopril, lisinopril, moexipril, perindopril, quinapril, ramipril, and trandolapril, all in free or pharmaceutically acceptable salts.

Especially preferred ACEIs for use in the present invention are benazepril, captopril, enalapril, quinapril and lisinopril, all in free or pharmaceutically acceptable salt form, for example benazepril HCl or enalapril maleate.

Anti-obesity agents are described below.

Such agents may be selected from the group consisting of CART (cocaine amphetamine regulated transcript) agonists, catecholaminergic agents (e.g. diethylpropion, phentermine, phenylpropanolamine, mazindol), NPY (neuropeptide Y) antagonists, MC 4 (melanocortin 4) agonists, MC 3 (melanocortin 3) agonists, orexin antagonists, TNF (tumor necrosis factor) agonists, CRF (corticotropin releasing factor) agonists, CRF BP (corticotropin releasing factor binding protein) antagonists, urocortin agonists, a melanin concentrating hormone antagonists, β3 adrenergic receptor agonists, MSH (melanocyte-stimulating hormone) agonists or mimetics, MCH (melanocyte-concentrating hormone) antagonists, thyromimetic agents, dehydroepiandrosterone or an analog thereof, glucocorticoid receptor agonist or antagonist, ciliary neurotrophic factors, human agouti-related protein antagonists, CCK (cholecystokinin) agonists, monoamine re-uptake inhibitors, serotonin re-uptake inhibitors, serotonin and noradrenaline re-uptake inhibitors, mixed serotonin and noradrenergic compounds, 5HT (serotonin) agonists, dopamine agonists, bombesin agonists, galanin antagonists, growth hormone, growth factors such as prolactin or placental lactogen, growth hormone releasing compounds, TRH (thyreotropin releasing hormone) agonists, UCP 2 or 3 (uncoupling protein 2 or 3) modulators, leptin agonists, DA agonists (bromocriptin, doprexin), lipase/amylase inhibitors, RXR (retinoid X receptor) modulators, TR β agonists, AGRP (Agouti related protein) inhibitors, opioid antagonists (such as naltrexone), exendin-4, PACAP (pituitary adenylyl cyclase activationg peptide), cannabinoid receptor antagonists, GLP-1 and ciliary neurotrophic factor.

Preferred anti-obesity agents are selected from the group consisting of phentermine, leptin, bromocriptine, dexamphetamine, amphetamine, fenfluramine, dexfenfluramine, sibutramine, orlistat, dexfenfluramine, mazindol, phentermine, phendimetrazine, diethylpropion, fluoxetine, bupropion, topiramate, diethylpropion, benzphetamine, phenylpropanolamine or ecopipam, ephedrine, pseudoephedrine and pharmaceutical salts thereof.

More preferred anti-obesity agents are selected from the group consisting of orlistat, sibutramine, diethylpropion, phen-fen and phentermine.

Preferably, the antidiabetic compound is selected from the group consisting of insulin signalling pathway modulators, like inhibitors of protein tyrosine phosphatases (PTPases), non-small molecule mimetic compounds and inhibitors of glutamine-fructose-6-phosphate amidotransferase (GFAT), compounds influencing a dysregulated hepatic glucose production, like inhibitors of glucose-6-phosphatase (G6Pase), inhibitors of fructose-1,6-bisphosphatase (F-1,6-BPase), inhibitors of glycogen phosphorylase (GP), glucagon receptor antagonists and inhibitors of phosphoenolpyruvate carboxykinase (PEPCK), pyruvate dehydrogenase kinase (PDHK) inhibitors, insulin sensitivity enhancers, insulin secretion enhancers, α-glucosidase inhibitors, inhibitors of gastric emptying, insulin, and α₂-adrenergic antagonists, or the pharmaceutically acceptable salts of such a compound and optionally at least one pharmaceutically acceptable carrier; for simultaneous, separate or sequential use, particularly in the prevention, delay of progression or treatment of conditions mediated by DPP-IV, in particular conditions of impaired glucose tolerance (IGT), conditions of impaired fasting plasma glucose, metabolic acidosis, ketosis, arthritis, obesity and osteoporosis, and preferably diabetes, especially type 2 diabetes mellitus. Such a combination is preferably a combined preparation or a pharmaceutical composition. Preferred DPP-IV inhibitors are N-(N′-substituted glycyl)-2-cyanopyrrolidines. Most preferred DPP-IV inhibitors are(S)-1-{2-[5-cyanopyridin-2-yl)amino]ethyl-aminoacetyl}-2-cyano-pyrrolidine (DPP728) or (S)-1-[(3-hydroxy-1-adamantyl)amino]acetyl-2-cyano-pyrrolidine (LAF237). The insulin sensitivity enhancer is preferably selected from the group consisting of antidiabetic thiazolidinediones, antidiabetic vanadium containing compounds and metformin.

In one preferred embodiment, the insulin sensitivity enhancer is metformin. Beta-blockers suitable for use in the present invention include beta adrenergic blocking agents (beta-blockers) which compete with epinephrine for beta-adrenergic receptors and interfere with the action of epinephrine. Preferably, the beta-blockers are selective for the beta adrenergic receptor as compared to the alpha adrenergic receptors, and so do not have a significant alpha-blocking effect. Suitable beta-blockers include compounds selected from acebutolol, atenolol, betaxolol, bisoprolol, carteolol, esmolol, labetalol, metoprolol, nadolol, oxprenolol, penbutolol, pindolol, propranolol, sotalol, and timolol. Where the beta-blocker is an acid or base or otherwise capable of forming pharmaceutically acceptable salts or prodrugs, these forms are considered to be encompassed herein, and it is understood that the compounds may be administered in free form or in the form of a pharmaceutically acceptable salt or a prodrug such as a physiologically hydrolizable and acceptable ester. For example, metoprolol is suitably administered as its tartrate salt, propranolol is suitably administered as the hydrochloride salt, and so forth

Especially preferred beta-blockers for use in the present invention are atenolol, metoprolol and propranolol.

The therapeutic agents which may be combined with valsartan include, but are not limited to anti-obesity agents selected from the group consisting of phentermine, leptin, bromocriptine, dexamphetamine, amphetamine, fenfluramine, dexfenfluramine, sibutramine, orlistat, dexfenfluramine, mazindol, phentermine, phendimetrazine, diethylpropion, fluoxetine, bupropion, topiramate, diethylpropion, benzphetamine, phenylpropanolamine or ecopipam, ephedrine, pseudoephedrine, antidiabetics, loop diuretics, such as ethacrynic acid, furosemide and torsemide; angiotensin converting enzyme (ACE) inhibitors such as benazepril, captopril, enalapril, fosinopril, lisinopril, moexipril, perinodopril, quinapril, ramipril and trandolapril; inhibitors of the Na—K-ATPase membrane pump such as digoxin; neutralendopeptidase (NEP) inhibitors; ACE/NEP inhibitors, such as omapatrilat, sampatrilat and fasidotril; beta adrenergic receptor blockers, such as acebutolol, atenolol, betaxolol, bisoprolol, metoprolol, nadolol, propranolol, sotalol and timolol; inotropic agents, such as digoxin, dobutamine and milrinone; calcium channel blockers, such as amlodipine, bepridil, diltiazem, felodipine, nicardipine, nimodipine, nifedipine, nisoldipine and verapamil; and 3-hydroxy-3-methyl-glutaryl coenzyme A reductase (HMG-CoA) inhibitors, such as lovastatin, pitavastatin, simvastatin, pravastatin, cerivastatin, mevastatin, velostatin, fluvastatin, dalvastatin, atorvastatin, rosuvastatin, fluidostatin and rivastatin.

The active agents of the present invention may be administered either simultaneously, before or after the other active ingredient, either separately by the same or different route of administration or together in the same pharmaceutical formulation.

The compounds of the invention depending on the nature of the substituents, may possess one or more asymmetric centers. The resulting diastereoisomers, enantiomers and geometric isomers are encompassed by the instant invention.

Depending on the choice of starting materials and methods, the compounds may be in the form of one of the possible isomers or mixtures thereof, e.g., as substantially pure geometric (cis or trans) isomers, optical isomers (antipodes), racemates, or mixtures thereof. The aforesaid possible isomers or mixtures thereof are within the purview of this invention. Any resulting mixtures of isomers can be separated on the basis of the physico-chemical differences of the constituents, into the pure geometric or optical isomers, diastereoisomers, racemates, e.g., by chromatography and/or fractional crystallization.

Any resulting racemates of final products or intermediates can be resolved into the optical antipodes by known methods, e.g., by separation of the diastereoisomeric salts thereof, obtained with an optically active acid or base, and liberating the optically active acidic or basic compound. The carboxylic acid intermediates can thus be resolved into their optical antipodes, e.g., by fractional crystallization of D- or L-(α-methylbenzylamine, cinchonidine, cinchonine, quinine, quinidine, ephedrine, dehydroabietylamine, brucine or strychnine)-salts. Racemic products can also be resolved by chiral chromatography, e.g., high pressure liquid chromatography using a chiral adsorbent.

Compounds of the invention are either obtained in the free form, or as a salt thereof if salt forming groups are present.

Acidic compounds of the invention may be converted into salts with pharmaceutically acceptable bases, e.g., an aqueous alkali metal hydroxide, advantageously in the presence of an ethereal or alcoholic solvent, such as a lower alkanol. From the solutions of the latter, the salts may be precipitated with ethers, e.g., diethyl ether. Resulting salts may be converted into the free compounds by treatment with acids. These or other salts can also be used for purification of the compounds obtained.

Compounds of the invention having basic groups can be converted into acid addition salts, especially pharmaceutically acceptable salts. These are formed, e.g., with inorganic acids, such as mineral acids, e.g., sulfuric acid, a phosphoric or hydrohalic acid; or with organic carboxylic acids, such as C₁-C₄alkanecarboxylic acids which, e.g., are unsubstituted or substituted by halogen, e.g., acetic acid, such as saturated or unsaturated dicarboxylic acids, e.g., oxalic, succinic, maleic or fumaric acid, such as hydroxy-carboxylic acids, e.g., glycolic, lactic, malic, tartaric or citric acid, such as amino acids, e.g., aspartic or glutamic acid; or with organic sulfonic acids, such as C₁-C₄alkyl-sulfonic acids, e.g., methanesulfonic acid, or arylsulfonic acids which are unsubstituted or substituted, e.g., by halogen. Preferred are salts formed with hydrochloric acid, methanesulfonic acid and maleic acid.

In view of the close relationship between the free compounds and the compounds in the form of their salts, whenever a compound is referred to in this context, a corresponding salt is also intended, provided such is possible or appropriate under the circumstances.

The compounds, including their salts, can also be obtained in the form of their hydrates, or include other solvents used for their crystallization.

Another aspect of the invention includes pharmaceutical compositions comprising a therapeutically effective amount of valsartan, either alone or combined with another therapeutic agent, and a pharmaceutically acceptable carrier.

Another aspect of the invention includes pharmaceutical compositions comprising a therapeutically effective amount of valsartan, either alone or combined with at least another therapeutic agent selected from the group of anti-hypertensive agents, anti-obesity agents, anti-diabetic agents, beta-blockers, inotropic agents and hypolipidemic agents.

In another embodiment, the present invention relates to a pharmaceutical composition comprising valsartan, alone or in combination with at least another therapeutic agent in the presence of a pharmaceutically acceptable carrier for the prevention or treatment of AF in patients having symptomatic heart failure.

In another embodiment, the present invention relates to the use of a pharmaceutical composition comprising valsartan, alone or in combination with at least another therapeutic agent in the presence of a pharmaceutically acceptable carrier for the preparation of a medicament for the prevention or treatment of AF in patients having symptomatic heart failure.

In a preferred embodiment therapeutic agent of the pharmaceutical composition according to the present invention is selected from the group consisting of anti-obesity agents selected from the group consisting of phentermine, leptin, bromocriptine, dexamphetamine, amphetamine, fenfluramine, dexfenfluramine, sibutramine, orlistat, dexfenfluramine, mazindol, phentermine, phendimetrazine, diethylpropion, fluoxetine, bupropion, topiramate, diethylpropion, benzphetamine, phenylpropanolamine or ecopipam, ephedrine, pseudoephedrine, antidiabetics, loop diuretics, such as ethacrynic acid, furosemide and torsemide; angiotensin converting enzyme (ACE) inhibitors such as benazepril, captopril, enalapril, fosinopril, lisinopril, moexipril, perinodopril, quinapril, ramipril and trandolapril; inhibitors of the Na—K-ATPase membrane pump such as digoxin; neutralendopeptidase (NEP) inhibitors; ACE/NEP inhibitors, such as omapatrilat, sampatrilat and fasidotril; beta adrenergic receptor blockers, such as acebutolol, atenolol, betaxolol, bisoprolol, metoprolol, nadolol, propranolol, sotalol and timolol; inotropic agents, such as digoxin, dobutamine and milrinone; calcium channel blockers, such as amlodipine, bepridil, diltiazem, felodipine, nicardipine, nimodipine, nifedipine, nisoldipine and verapamil; and 3-hydroxy-3-methyl-glutaryl coenzyme A reductase (HMG-CoA) inhibitors, such as lovastatin, pitavastatin, simvastatin, pravastatin, cerivastatin, mevastatin, velostatin, fluvastatin, dalvastatin, atorvastatin, rosuvastatin, fluidostatin and rivastatin.

In a preferred embodiment, the invention relates to pharmaceutical compositions comprising a therapeutically effective amount of valsartan combined with at least an anti-hypertensive agent consisting of an ACEI selected from the group consisting of benazepril, captopril, cilazapril, enalapril, enalaprilat, fosinopril, lisinopril, moexipril, perindopril, quinapril, ramipril, and trandolapril, all in free or pharmaceutically acceptable salts.

In a preferred embodiment, the invention relates to pharmaceutical compositions comprising a therapeutically effective amount of valsartan combined with at least an anti-obesity agent selected from the group consisting of phentermine, leptin, bromocriptine, dexamphetamine, amphetamine, fenfluramine, dexfenfluramine, sibutramine, orlistat, dexfenfluramine, mazindol, phentermine, phendimetrazine, diethylpropion, fluoxetine, bupropion, topiramate, diethylpropion, benzphetamine, phenylpropanolamine or ecopipam, ephedrine and pseudoephedrine and pharmaceutical salts thereof.

In a preferred embodiment, the invention relates to pharmaceutical compositions comprising a therapeutically effective amount of valsartan combined with at least an anti-diabetic agent selected from the group consisting of phentermine, leptin, bromocriptine, dexamphetamine, amphetamine, fenfluramine, dexfenfluramine, sibutramine, orlistat, dexfenfluramine, mazindol, phentermine, phendimetrazine, diethylpropion, fluoxetine, bupropion, topiramate, diethylpropion, benzphetamine, phenylpropanolamine or ecopipam, ephedrine, pseudoephedrine and pharmaceutical salts thereof.

In a preferred embodiment, the invention relates to pharmaceutical compositions comprising a therapeutically effective amount of valsartan combined with at least a beta-blocker agent selected from the group consisting of atenolol, metoprolol and propranolol.

In a preferred embodiment, the invention relates to pharmaceutical compositions comprising a therapeutically effective amount of valsartan combined with at least an ACEI selected from the group consisting of benazepril, captopril, cilazapril, enalapril, enalaprilat, fosinopril, lisinopril, moexipril, perindopril, quinapril, ramipril, and trandolapril, all in free or pharmaceutically acceptable salts and at least a beta-blocker agent selected from the group consisting of atenolol, metoprolol and propranolol.

In a preferred embodiment, the invention relates to pharmaceutical compositions comprising a therapeutically effective amount of valsartan combined with at least an inotropic agent.

In a preferred embodiment, the invention relates to pharmaceutical compositions comprising a therapeutically effective amount of valsartan combined with at least an hypolipidemic agent.

In a preferred embodiment, the invention relates to pharmaceutical compositions comprising a therapeutically effective amount of valsartan combined with an ACE inhibitor and a beta-blocker

The pharmaceutical compositions according to the invention can be prepared in a manner known per se and are those suitable for enteral, such as oral or rectal, and parenteral administration to mammals (warm-blooded animals), including man, comprising a therapeutically effective amount of the pharmacologically active compound, alone or in combination with one or more pharmaceutically acceptable carriers, especially suitable for enteral or parenteral application. Typical oral formulations include tablets, capsules, syrups, elixirs and suspensions. Typical injectable formulations include solutions and suspensions. The pharmaceutical compositions may be employed to preventing or reducing the incidence of AF and thereby reduce the risk of morbidity and mortality in patients having symptomatic heart failure.

The typical pharmaceutically acceptable carriers for use in the formulations described above are exemplified by sugars such as lactose, sucrose, mannitol and sorbitol; starches, such as cornstarch, tapioca starch and potato starch; cellulose and derivatives, such as sodium carboxymethyl cellulose, ethyl cellulose and methyl cellulose; calcium phosphates, such as dicalcium phosphate and tricalcium phosphate; sodium sulfate; calcium sulfate; polyvinylpyrrolidone; polyvinyl alcohol; stearic acid; alkaline earth metal stearates, such as magnesium stearate and calcium stearate; stearic acid; vegetable oils, such as peanut oil, cottonseed oil, sesame oil, olive oil and corn oil; non-ionic, cationic and anionic surfactants; ethylene glycol polymers; betacyclodextrin; fatty alcohols; and hydrolyzed cereal solids, as well as other non-toxic compatible fillers, binders, disintegrants, buffers, preservatives, antioxidants, lubricants, flavoring agents and the like commonly used in pharmaceutical formulations.

These pharmaceutical preparations are for enteral, such as oral, and also rectal or parenteral, administration to homeotherms, with the preparations comprising the pharmacological active compound either alone or together with customary pharmaceutical auxiliary substances. For example, the pharmaceutical preparations consist of from about 0.1-90%, preferably of from about 1% to about 80%, of the active compounds. Pharmaceutical preparations for enteral or parenteral administration are, e.g., in unit dose forms, such as coated tablets, tablets, capsules or suppositories and also ampoules. These are prepared in a manner which is known per se, e.g., using conventional mixing, granulation, coating, solubulizing or lyophilizing processes. Thus, pharmaceutical preparations for oral use can be obtained by combining the active compounds with solid excipients, if desired granulating a mixture which has been obtained, and, if required or necessary, processing the mixture or granulate into tablets or coated tablet cores after having added suitable auxiliary substances.

While the precise dosage will vary depending on the individual patient, and some adjustment by the treating physician may be required, suitable dosages of valsartan are generally as known in the art. For example, in the method of the invention, valsartan is preferably administered to adult patients once (o.d.) or twice daily (b.i.d.) for a total daily dosage of 20-320 mg, preferably 80-320 mg, preferably as the free acid.

The pharmaceutical compositions for use in the present invention are preferably compositions for oral administration as are known and commercially available from the manufacturers. Suitable compositions and information concerning suitable pharmaceutically effective dosages and potential side effects are described in the Physician's Desk Reference. The precise dosage of the active compounds can depend on a variety of factors, such as mode of administration, age and/or individual condition. Where an active agent is an acid or base or otherwise capable of forming pharmaceutically acceptable salts or prodrugs, these forms are considered to be encompassed herein, and it is understood that the compounds may be administered in free form or in the form of a pharmaceutically acceptable salt or a prodrug such as a physiologically hydrolizable and acceptable ester, especially Where the salt or prodrug form is the form approved by the regulatory authorities and commonly available.

Valsartan is supplied in the form of suitable dosage unit form, e.g., a capsule or tablet, in free or pharmaceutically acceptable salt form, comprising a therapeutically effective amount, e.g., an amount equivalent to from about 20 mg to about 320 mg of valsartan as free acid. The administration of the active ingredient may occur up to three times a day (t.i.d.), starting, e.g., with a daily dose of 20 mg or 40 mg of valsartan, increasing via 80 mg daily and further to 160 mg daily up to 320 mg daily. Preferably, valsartan is administered o.d. or b.i.d. to patients with a dose of 80 mg or 160 mg, for a total daily dose of 20-320 mg, preferably 80-320 mg/day. Corresponding doses may be taken, e.g., in the morning, at mid-day or in the evening.

It has been found that administration to a patient having heart failure of valsartan, or pharmaceutically acceptable salts thereof, alone or in combination with a therapeutically effective amount of at least another therapeutic agentoptionally in the presence of a pharmaceutically acceptable carrier reduces the occurrence of AF and thereby reduces the risk of morbidity and mortality associated therewith.

The occurrence of AF was evaluated based on adverse event reports in the patients with symptomatic heart failure (ejection fraction<40% and dilated ventricle-left ventricular internal diastolic diameter (LVIDD)>2.9) enrolled in the Val-HeFT trial. The Val-HeFT trial is set forth in Example 2.

The following examples illustrate the above-described invention; however, it is not intended to restrict the scope of this invention in any manner. All publications and patents mentioned herein are incorporate by reference in their entirety as if set forth in full herein.

### EXAMPLE 1

The occurrence of AF was evaluated based on adverse event reports in the patients with symptomatic HF (EF<40% and dilated ventricle-LVIDD>2.9) enrolled in the Val-HeFT trial (set forth in Example 2). Patients were randomized to valsartan or placebo on top of the prescribed treatments for heart failure. During the 23 months of follow-up of the study, AF was reported in 328/5000 patients (6.6%), 132/2506 (5.27%) in the valsartan allocated patients and 196/2494 (7.86%) in those allocated to placebo (p=0.0002). Logistic regression analysis showed that ischemic etiology of heart failure (RR: 1.35; 95% confidence interval (CI): 1.06-1.71), to be older than 70 years (RR: 1.49; 95% CI: 1.17-1.89), and the valsartan treatment (RR: 0.65; 95% CI: 0.52-0.82) were independently associated with AF occurrence. Cox multivariate regression analysis showed that occurrence of AF was independently associated with a worse prognosis: 23-month all-cause mortality was 30.2% and 18.8%, respectively, in patients with and without AF occurrence (RR: 1.43; 95% CI: 1.16-1.76).

The results demonstrate that adding valsartan to prescribed therapy as shown in Example 2 (93% ACE-inhibitors, 35% beta-blockers) significantly prevents or reduces the incidence of AF by nearly 35%.

### EXAMPLE 2

1. Introduction

CHF is a complex clinical syndrome with varying pathophysiology and clinical expression. It is well-known that overt heart failure is characterized by activation of the RAS and other neuroendocrine systems [1, 2]. Increased activity of the RAS is considered to be responsible for vasoconstriction, sodium retention with volume expansion, norepinephrine release from cardiac sympathetic nerves and cardiac hypertrophy. Due to loss of compensatory mechanisms in heart failure patients, these pathological processes result in progressive left ventricular dysfunction [3].

ACE inhibitors block the formation of Ang II from angiotensin I (Ang I) and thus are expected to suppress the deleterious action of a stimulated RAS. In clinical trials, ACE inhibitors were found to lower blood pressure, improve left ventricular hypertrophy, reduce morbidity and mortality in congestive cardiac failure, and prevent progression to overt cardiac failure in patients with depressed ventricular function or myocardial infarction [4]. Despite this advance in pharmacological therapy, mortality is high, with approximately 50% of patients with heart failure dying within 5 years of diagnosis [5].

There are, however, non-ACE enzymatic pathways for the formation of Ang II that are not blocked by ACE inhibitors-notably the enzyme cardiac chymase [6,7]. These alternative pathways may be especially important in the tissue formation of Ang II, so that inhibition of the RAS by ACE inhibitors is incomplete. In a study on left ventricular function in patients with heart failure taking ACE inhibitors, patients who ultimately deteriorated were found to have higher levels of Ang II than patients who remained stable [8].

The efficacy of ACE inhibitors may also be limited by the fact that they are competitive inhibitors. Thus, high levels of Ang I resulting from ACE inhibition might drive continued production of Ang II; or suppression of Ang II might upregulate the Ang II receptor, thus increasing the sensitivity to Ang II.

Furthermore, ACE, also known as kininase II, is not a very specific enzyme and has other possible substrates besides Ang I, such as bradykinin. Increased bradykinin levels which are thought to be associated with the use of ACE inhibitors, may have important physiologic effects that are potentially beneficial as well as detrimental as in the case of ACE inhibitor-induced dry cough [9].

Taken together, the proven beneficial effects of ACE inhibitors in cardiovascular diseases can be attributed to at least partial suppression of the formation of Ang II. The contribution of increased bradykinin levels to the beneficial effects seen with ACE inhibitors remains controversial at present. A more specific and complete inhibition of the RAS, e.g., at the Ang II receptor level, alone, or in combination with ACE inhibition, is expected to result in further clinical benefit.

Valsartan (CGP 48933) is an orally active, potent and specific competitive Ang II antagonist at the level of the AT₁-receptor subtype [10]. Treatment of patients with essential arterial hypertension with valsartan 80 mg o.d. produced decreases in systolic and diastolic blood pressure comparable to those achieved by treatment with ACE inhibitors and other antihypertensive medications, with a more favorable tolerability profile for valsartan.

The effect of valsartan alone and in combination with an ACE inhibitor was studied in a model of rapid pacing induced heart failure in mini pigs. The results of this study suggest that combined ACE inhibition and AT₁-receptor blockade provides further beneficial effects on left ventricular function and geometry, and myocyte inotropic response, when compared to either ACE inhibition or AT₁-receptor blockade alone.

Two Phase II studies (Protocols 103 and 104) have been recently completed. Protocol 103 is a randomized, double-blind, placebo-controlled, parallel group, dose ranging trial to determine the chronic central hemodynamic effects of valsartan 40 mg b.i.d., 80 mg b.i.d., 160 mg b.i.d. and lisinopril 10 mg o.d. administered for 4 weeks to non-ACE inhibitor treated CHF patients (NYHA Class II-IV). Preliminary trial results show that valsartan 40, 80, and 160 mg b.i.d. produced clinically relevant and statistically significant improvements in cardiac hemodynamics versus placebo.

Protocol 104 is a randomized, double-blind, placebo-controlled, parallel group trial to determine the chronic central hemodynamic effects of valsartan 80 mg b.i.d. and 160 mg b.i.d. administered for 4 weeks to ACE inhibitor treated CHF patients (NYHA Class II-IV). The targeted sample size was 75 completed patients. Trial results are pending. Twice daily dosing was chosen in Valsartan Protocols 103 and 104 to maximize detecting a clinically significant hemodynamic effect in the selected patient populations.

In this placebo-controlled trial, the effect of valsartan on morbidity and mortality will be determined in patients with stable, chronic congestive heart failure (NYHA Class II-IV) receiving ACE inhibitors and those patients where ACE inhibitors are contraindicated. Also, the effect of valsartan on signs and symptoms of CHF and quality of life will be determined.

2. Trial Objective(s)

To assess the effect of valsartan, in comparison with placebo, on morbidity and mortality, signs and symptoms, and quality of life in patients with stable, chronic congestive heart failure (NYHA Class II-IV).

3. Investigational Plan

3.1. Trial Design and Design Rationale

Design

This is a multicountry, randomized, double-blind, forced titration, parallel, placebo-controlled trial. Patients will be stratified according to their use of beta blockers at randomization.

Schematic design diagram
Flow chart Valsartan Protocol 107

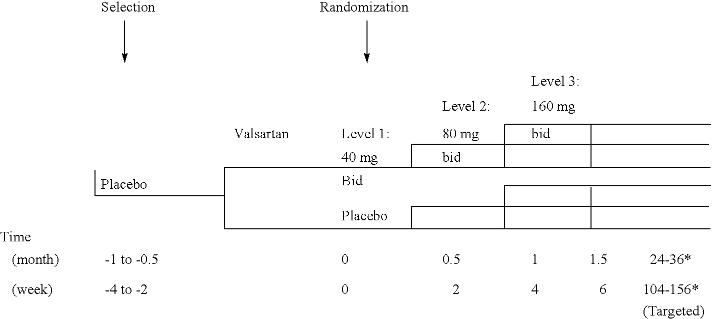

*The trial will continue and all randomized patients will remain in the trial until 906 deaths have occurred or until statistically significant results are observed for either of the two interim analyses. The actual trial duration will depend on the interim analysis results, patient accrual rate, length of patient accrual period, and observed death rates.

Design Rationale
Trial design
   The double-blind, randomized, placebo-controlled, parallel design has been shown to reduce bias in comparing different treatment regimens with respect to efficacy and safety. Randomized treatment allocation will be stratified by baseline (Visit 2) beta blocker/no-beta blocker background therapy to obtain as much similarity as possible between the two treatment groups with respect to this background therapy.
Dose and dose interval
   Twice daily dosing of valsartan allows maximum exposure to study drug in a manner demonstrated to be safe in Phase II trials.
   Forced titration is used:
   (i) to up-titrate the majority of patients to the,high dose of valsartan 160 mg b.i.d. and thus theoretically maximize the number of patient responders to valsartan;
   (ii) to allow patients with problems tolerating the high dose to remain in the trial at presumed therapeutic doses; and
   (iii) to allow gradual exposure to the valsartan 160 mg b.i.d. dose.
Population
   The target population for treatment is that of symptomatic patients with congestive heart failure NYHA Class II-IV. This patient population is characterized by activation of the renin-angiotensin system which in turn is considered to be, at least partially, responsible for the high mortality (overall 12% within one year) despite the beneficial effects of currently available treatments.
   The trial population, as defined in the inclusion/exclusion criteria, is expected to be somewhat more homogeneous, to control variability in treatment comparisons, but otherwise representative of the targeted treatment population.
Controls/Comparators
   The allowance of the use of standard CHF background therapy is made for ethical and for practical reasons.
   In this trial, patients will receive either valsartan or placebo in addition to standard CHF background therapy. It is not expected that placebo will have an effect on morbidity or mortality in this trial. The inclusion of a placebo treatment arm will allow for a direct, concurrent comparison of valsartan and placebo which should provide an objective measure for the effectiveness of the drug.
Blinding
   Patients will be blinded during the single-blind placebo run-in period and patients and investigators will be blinded during the double-blind active treatment period. In addition, the treatment codes will remain blinded to all Ciba (or other contracted) personnel involved in monitoring the trial until after the database is locked for final analysis. These blinding conditions will reduce the chance of introducing bias during data collection and monitoring.
Measures
   The efficacy of valsartan will be determined using validated measures for diagnosis and progression of CHF, i.e., recording of deaths; recording of morbid events as defined in Section 5.2.1; detailed history and physical examination for signs and symptoms of CHF including ECG, ejection fraction and left ventricular internal diastolic diameter; and Minnesota Living with Heart Failure Questionnaire for quality of life.
   The pharmacoeconomic assessments performed in this trial will be used to provide economic data and will be described in a separate trial protocol and reported separately.

Sample Size

A total of 906 patient deaths (which occur while the patient is still on double-blind trial medication, i.e., prior to the patient's permanent discontinuation of double-blind trial medication) is required for the trial (unless statistically significant interim analysis results occur beforehand). The numbers of randomized and completed patients needed to achieve 906 patient deaths will be variable, depending on patient accrual rate, length of accrual period, total length of trial after first randomization, and observed death rates.

For planning purposes, assuming uniform enrollment over a 1-year period and total trial duration of 3 years after randomizing the first patient, a total of approximately 3,660 completed patients (1,830 per treatment group) is needed.

All randomized patients (including patients with morbid events other than death) should remain in the trial on double-blind medication until death or until trial end, except in case of the events described in Section 3.7. Assuming a post-randomization double-blind-treatment discontinuation rate of around 15% for these events, a total of approximately 4,310 randomized patients (2,155 per treatment group) is needed. The actual numbers of completed and randomized patients needed to achieve 906 patient deaths will be assessed on an on-going basis during the trial.

Further discussion regarding sample size calculation is given in Section 5.1.

Amendments to the Protocol

Any amendment to the trial that seems appropriate as the trial progresses will be agreed upon between the Steering Committee of the trial and Ciba. Amendments will be submitted to the ERB/IRB for written approval and then made a formal part of the protocol, before implementation. The expedited review procedure for an amendment (if available in a specific country) is appropriate only if minor changes are made in the protocol which do not alter patient risk. The written signed approval from the ERB/IRB should refer specifically to the investigator and to the protocol number and title and mention any amendment numbers that are applicable.

Monitoring Committees

The following boards and committees will be installed for this trial:
Data and Safety Monitoring Board
Endpoint Committee
Steering Committee
Executive Committee

A detailed description on the function and membership of the individual boards/committees is given in Appendix 7.4.

Two interim analyses and one final analysis are planned for this trial. The analyses, the circumstances regarding trial termination, and the statistical adjustments used for these analyses are described in Section 5.

Duration of Trial

The single-blind placebo run-in period will be 2 to 4 weeks.

Unless completed early because of statistically significant interim analysis results, the trial will continue until 906 deaths, occurring prior to permanent discontinuation of trial medication, have been observed. Therefore, the duration of the double-blind period is variable. The targeted duration of the double-blind treatment period is 24 to 36 months for each patient unless a patient prematurely discontinues from the trial (see Section 3.7). The scheduled time allotted for patient enrollment is 12 months. Accordingly, the targeted total trial durations for the first and last patients enrolled are 37 months (first patient) and 25 months (last patient).

Possible adjustments to the enrollment period may be made during the trial in order to facilitate trial completion within 3 years.

Additional discussion regarding trial completion is given in Section 5.1.

3.2. Trial Population

3.2.1. Inclusion Criteria
Males or females; minimum 18 years of age, with congestive heart failure (NYHA Class II-IV) beginning at least 3 months prior to Visit 1. Females must be post-menopausal for one year, surgically sterilized or using effective forms of contraception, i.e., abstinence, hormonal contraceptive, IUD or barrier method with spermicide, and with negative pregnancy tests throughout the trial.
Ejection fraction<40% on echocardiography and left ventricular internal diameter in diastole>2.9 cm/m² on echocardiography within one week prior to Visit 1 or during the placebo run-in period.
Each patient's heart failure medication must remain at a stable dosage regimen for two weeks prior to Visit 1 and during the placebo run-in period.
The patient must indicate a willingness to participate by providing written informed consent.

3.2.2. Exclusion Criteria
Pregnancy, nursing or women of childbearing potential not practicing effective contraceptive methods
Right heart failure due to pulmonary disease
Diagnosis of postpartum cardiomyopathy
Presence of rapidly deteriorating heart failure
Myocardial infarction or cardiac surgery, including percutaneous transluminal coronary angioplasty (PTCA), within past 3 months
Patients with a history of heart transplant or who are on a transplant list
Unstable angina or coronary artery disease likely to require coronary artery bypass graft (CABG) or PTCA
Sustained ventricular arrhythmia with syncopal episodes within past 3 months that is untreated
Presence of hemodynamically significant mitral stenosis or mitral regurgitation, except mitral regurgitation secondary to left ventricular dilatation
Presence of hemodynamically significant obstructive lesions of left ventricular outflow tract, including aortic stenosis
Persistent standing systolic blood pressure<90 mm Hg
Stroke within the past 3 months
Primary liver disease considered to be life-threatening
Renal disease likely to be life threatening or serum creatinine>2.5 mg/dL
Malignancies likely to limit 5 year survival
History or presence of any other diseases with a life expectancy of<5 years
Contraindication to use angiotensin II receptor antagonists
Prior or current double-blind treatment in valsartan CHF trials
Participation in an investigational drug study within the past 30 days
Any condition that in the opinion of the investigator or medical monitor would jeopardize the evaluation of efficacy or safety
History of noncompliance to medical regimens and patients who are considered potentially unreliable.

Treatment with any of the following drugs within the past 3 months prior to Visit 1:
Class IC antiarrhythmic agents, such as flecanide and propafenone
Chronic intermittent intravenous inotrope or intravenous vasodilator therapy
Angiotensin II receptor antagonists including valsartan

3.3. Treatment Allocation

3.3.1. Patient Numbering

At Visit 1, patients will be assigned a sequential Patient Number ranging from 0001-5000 for U.S. trial centers and from 10001-15000 for non-U.S. trial centers.

At randomization (Visit 2), patients in stratum 1 (without beta blocker background therapy) will be assigned a sequential Randomization Number ranging from 5001-8000 for U.S. trial centers and from 15001-18000 for non-U.S. trial centers. For patients in stratum 2 (with beta blocker background therapy) sequential Randomization Numbers will range from 8001-9996 for U.S. trial centers and from 18001-20000 for non-U.S. trial centers.

3.3.2. Stratification

Patients will be stratified according to their use of beta blockers at randomization. Patients not taking beta blockers at randomization will be assigned to stratum 1 and patients taking beta blockers at randomization will be assigned to stratum 2.

3.4. Test Drugs

3.4.1. Dosage/Administration

Throughout the trial, daily dosing of trial medication will be one capsule in the morning (between 6:00 and 8:00 A.M.) and one capsule in the evening (between 6:00 and 8:00 P.M.). Patients will take their morning dose of trial medication at home on the day of their scheduled visit.

Single-blind Placebo Run-in Period

At Visit 1, eligible patients will enter a single-blind placebo run-in period for 2 to 4 weeks.

Double-blind Treatment Period

At Visit 2, patients who meet the randomization criteria will be stratified according to their use of beta blockers and randomized to receive either valsartan 40 mg b.i.d. or placebo (Level 1). After 2 weeks of treatment (Visit 3), patients will be titrated up to receive either valsartan 80 mg b.i.d. or placebo, respectively (Level 2). The criteria for titration are given below. Following another 2 week treatment period (Visit 4), patients will again be titrated up to receive valsartan 160 mg b.i.d. or placebo, respectively (Level 3). The criteria for titration are given below. Patients not tolerating the highest dose of valsartan will be titrated down to the next lower dose after 2 weeks of treatment (Visit 5).

Every effort should be made to maintain each patient at the highest tolerated titration dose level subsequent to Visit 5 for the duration of the double-blind period. If an intercurrent acute medical illness requires temporary interruption of trial medication, treatment should be reinstituted as soon as medically acceptable, preferably within 2 weeks of its cessation. If there is a lapse in therapy of greater than two months, the Ciba monitor/medical advisor should be notified.

Every effort should also be made to keep each patient's background medication constant subsequent to Visit 2 (randomization). The following guidelines should be followed with respect to background therapy:

1) The dose of digitalis, where applicable, should be reduced if there are signs of toxicity.

2) The dose of diuretics, where applicable, may be adjusted if the patient develops signs of either hypovolemia or positive fluid balance.

3) If the patient is euvolemic and consideration must be given to adjusting therapy with either ACEI, or hydralazine/nitrate, or trial treatment, e.g., hypotension, renal insufficiency, worsening symptoms of CHF, the investigator must determine a course of therapy best suited for the individual patient. In general, trial medication should be adjusted before background therapy is altered.

All randomized patients (including patients with morbid events other than death) will continue to receive double-blind treatment until death or until trial completion, except as noted in Section 3.7. The targeted length of the double-blind treatment period is 24-36 months and will vary from patient to patient.

Criteria for Titration

At Visits 3 and 4, titration to the next higher dose level will be done if all three of the following criteria are met:
persistent standing systolic blood pressure>90 mm Hg

AND
no symptoms of hypotension (syncope, faintness, orthostatic dizziness) are reported on current dose

AND
no increase in serum creatinine>50% from baseline (Visit 2).

At Visits 3, 4 or 5, patients with persistent standing systolic blood pressure<80 mm Hg OR symptoms of hypotension (syncope, faintness, orthostatic dizziness) OR increase in serum creatinine>50% from baseline (Visit 2) must be titrated down to the previous dose level or discontinued from trial treatment in case of the lowest possible dose level.

At Visits 3, 4 or 5, patients with standing systolic blood pressure>80 mm Hg and<90 mm Hg AND no symptoms of hypotension (syncope, faintness, orthostatic dizziness) AND no increase in serum creatinine>50% from baseline (Visit 2) will be maintained on their current dose level.

3.4.2. Blinding

All medication will be supplied as capsules of identical appearance.

The investigator site personnel as well as the Ciba personnel involved in the monitoring or conducting of the trial will be blinded to the trial drug codes. Trial drug codes will not be available to the above personnel until after the completion of the trial and final data review, except in the case of an emergency.

3.4.3. Breaking Treatment Codes

An individual decoding unit containing emergency identification of the package contents will be provided for each container of medication.

1. These decoding units are not to be opened unless an actual emergency occurs. The investigator also is to make a careful note of the date, time of opening, and the reason.

2. In the event that a decoding unit is opened, the Ciba monitor/MA (Medical Advisor) will be notified immediately by the investigator.

3. At the conclusion of the trial, all decoding units are to be returned to Ciba together with unused drug supplies and will be examined.

When the trial is completed, the data file verified, and protocol violations determined by the Ciba monitor/MA, drug codes will be broken and made available for data analysis.

3.4.4. Unused Medications

Where medication is dispensed to individual patients, they should be instructed to return the unused medication at each visit.

All unused trial medication must be returned to Ciba. Instructions for the return of trial medication will be provided by Ciba.

At each visit, with the exception of Visit 1, the number of unused and returned capsules per patient will be recorded on a drug accounting sheet.

Each time trial drug is dispensed to the patient a portion of the label on the bottle will be attached to the case report form. This label will be used to verify that the patient received the correct trial drug.

3.4.6. Supplies/Packaging

The following trial medication will be supplied:
Placebo capsules to be orally administered
Valsartan 40 mg capsules to be orally administered
Valsartan 80 mg capsules to be orally administered
Valsartan 160 mg capsules to be orally administered

Trial medication will be supplied in bottles. The trial drugs will be stored in a locked storage facility until they are returned to Ciba.

Trial Drug Supply per Patient

Up to, and including, Visit 7, the investigator will receive packed supplies for all possible dose levels for each randomized patient and visit (see diagram below).

Following Visit 7, the procedure for re-supply of trial medication will be different for US and non-US centers.

Re-supply of Trial Medication for US Centers

At Visit 7 (or within 2 days thereafter), the investigator will inform Ciba of the patient's designated dose level from Visit 7 onwards and will request re-supply for the next 12 months.

For Visit 8, the investigator will receive from Ciba the requested patient's dose level for 12 months (i.e. until/including Visit 11). Whenever the investigator changes the patient's designated dose level at or between Visits 8-11, he/she will use the required dose level from the unused patient medication from Visits 5, 6, and 7.

At Visit 11 (or within 2 days thereafter), the investigator will inform Ciba on the patient's designated dose level from Visit 12 onwards and will request re-supply for 12 months.

For Visit 12, the investigator will receive from Ciba the requested patient's dose level for 12 months (i.e. until/including Visit 15). Whenever the investigator changes the patient's designated dose level at or between Visits 12- 15, he/she will use the required dose level from the unused patient medication from Visits 5, 6, and 7.

At Visit 15 (or up to 2 days thereafter), the investigator will inform Ciba on the patient's designated dose level from Visit 16 onwards and will request re-supply for 12 months.

This process will continue annually. At or close to trial end, the amount of re-supply will be less than 12 months.

In cases where no more unused patient medication from Visits 5, 6, and 7 is available, Ciba will provide in addition to the requested dose level, the two alternative dose levels at the request of the investigator.

The investigator will request any re-supply from Ciba using a separate order sheet.

Each time the investigator dispenses trial medication to the patient, the tear-off label from the visit pack indicating the patient number and visit number will be attached to the CRF.

Re-supply of Trial Medication for Non-US Centers

Ciba will provide non-US centers with coded trial medication for all three dose levels.

At Visit 8, the investigator will contact Ciba and request the code for the patient's designated dose level. Using the code number, the investigator will then identify the patient's trial medication from the provided coded trial medication for the next 12 months (until/including Visit 11). Whenever the investigator changes the patient's designated dose level at or between Visits 8-11, he/she will contact Ciba and request the code for the patient's new designated dose level. Using the code number, the investigator will then identify the patient's new trial medication from the provided coded trial medication for the next 12 months.

At Visit 12, the investigator will contact Ciba again and request the code for the patient's designated dose level. Using the code number, the investigator will then identify the patient's trial medication from the provided coded trial medication for the next 12 months (until/including Visit 15). Whenever the investigator changes the patient's designated dose level at or between Visits 12-15, he/she will contact Ciba and request the code for the patient's new designated dose level. Using the code number, the investigator will then identify the patient's new trial medication from the provided coded trial medication for the next 12 months.

This process will continue annually. At or close to trial end, the amount of re-supply will be less than 12 months.

The investigator will request any re-supply from Ciba using a separate order sheet.

Each time the investigator dispenses trial medication to the patient, the tear-off label from the visit pack indicating the patient number and visit number will be attached to the CRF.

Packaging scheme

| Visit | Dose | Amount | Label |
|---|---|---|---|
| V 1 | Placebo (Level 1) | 76 | Patient-, visit - and level number |
| V 2 | Valsartan 40 mg or placebo (level 1) | 42 | Random.-, visit - and level |
| | | | number |
| V 3 | Valsartan 80 mg or placebo (level 2) + | 42 | Random.-, visit - and level |
| | Valsartan 40 mg or placebo (level 1) | | number |
| | | 42 | Random.-, visit - and level |
| | | | number |
| V 4 | Valsartan 160 mg or placebo (level 3) + | 42 | Random.-, visit - and level |
| | Valsartan 80 mg or placebo (level 2) + | | number |
| | Valsartan 40 mg or placebo (level 1) | 42 | Random.-, visit - and level |
| | | | number |
| | | 42 | Random.-, visit - and level |
| | | | number |
| V 5 | Valsartan 160 mg or placebo (level 3) + | 42 | Random.-, visit - and level |
| | Valsartan 80 mg or placebo (level 2) + | | number |
| | Valsartan 40 mg or placebo (level 1) | 42 | Random.-, visit - and level |
| | | | number |
| | | 42 | Random.-, visit - and level |
| | | | number |
| V 6 | Valsartan 160 mg or placebo (level 3) + | 152 | Random.-, visit - and level |
| | Valsartan 80 mg or placebo (level 2) + | | number |
| | Valsartan 40 mg or placebo (level 1) | 152 | Random.-, visit - and level |
| | | | number |
| | | 152 | Random.-, visit - and level |
| | | | number |
| V 7 | Valsartan 160 mg or placebo (level 3) + | 152 | Random.-, visit - and level |
| | Valsartan 80 mg or placebo (level 2) + | | number |
| | Valsartan 40 mg or placebo (level 1) | 152 | Random.-, visit - and level |
| | | | number |
| | | 152 | Random.-, visit - and level |
| | | | number |
| V 8 to | Re-supply on demand per patient and | 228 | Random.-, visit - and level |
| trial end | dose level in 3 month packs: | | number |
| | 3 month pack, e.g., 4 × 3 month packs of | | |
| | designated dose level | | |

3.5. Concomitant Treatments

Patients must receive stable pharmacological treatment of heart failure for at least two weeks prior to Visit 1 and during the placebo run-in period. In addition, every effort should be made to keep each patient's background CHF medication constant subsequent to randomization (see discussion Section 3.4.1.). Pharmacological treatment of heart failure includes the use of diuretics, ACE inhibitors, digoxin, hydralazine hydrochloride, and nitrates. Drugs for the treatment of arrhythmias (with the exception of Class IC agents such as flecainide and propafenone) are permitted and should follow a stable regimen. Acute and stable prophylactic antianginal treatment using nitroglycerin and derivatives is permitted. Any change in dose regimen of a concomitant medication for cardiovascular disease during the trial must be recorded in the CRF.

A low sodium diet, provided that it is administered regularly, is permitted.

3.6. Trial Procedures

Schematic Diagram

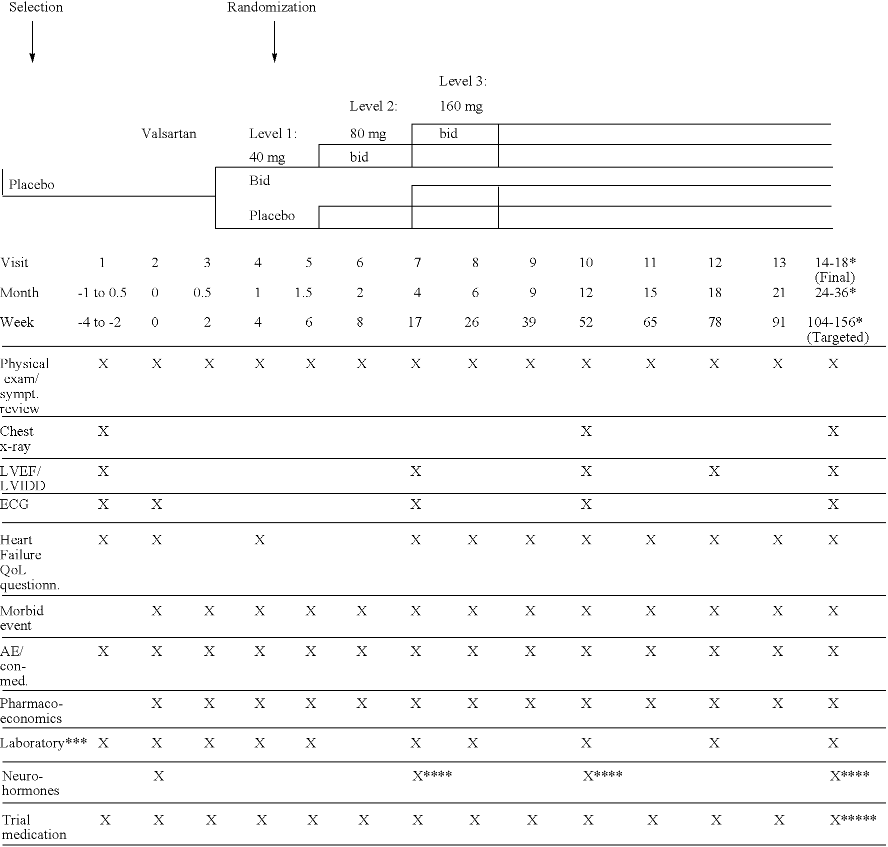

* = 3 month (13 week) Visit interval;
procedures at Visit 14 as shown;
procedures at Visit 15 and 17 as for Visit 11;
procedures at Visit 16 as for Visit 12;
procedures at Visit 18 as for Visit 10;
**The trial will continue until 906 deaths have been observed or statistically significant interim results
*** = hematology, blood chemistry, urinalysis, pregnancy test
****only Norepinephrine
***** = except final visit

3.6.1. Visit Procedures

All trial visits should be scheduled between 8:00 A.M. and 11:00 A.M., whenever possible.

To schedule trial visits, a half month will count as 2 weeks (14 days), one month will count as 4 weeks (28 days), 2 months will count as 9 weeks (63 days), and 3 months will count as 13 weeks (91 days).

The trial will continue until 906 deaths prior to permanent discontinuation of trial medication have been observed or statistically significant interim results are obtained. The targeted double-blind trial duration is 2 to 3 years with 14 to 18 visits for each patient. Depending on the actual event rate, the trial may proceed after Visit 18. In this section, visit procedures up to Visit 22 will be described.

Female patients of childbearing potential will have a pregnancy test at every visit. In case of a confirmed positive pregnancy test result, a patient who decides to carry a pregnancy to term, must be discontinued from trial treatment, but should still remain in the trial.

Patients who are permanently discontinued from double-blind trial treatment for any reason should continue to visit the investigator according to the protocol until trial end or death. In these cases, no trial medication will be dispensed to the patient and data collection will include: date of visit, date of occurrence of morbid event(s), and cardiovascular-related concomitant medications. If necessary, visits made after discontinuation from trial treatment may be conducted by phone. The date and principal reason for the patient's permanent discontinuation from double-blind trial medication must be collected and entered on the appropriate CRF.

The procedure for supply of trial medication is described in Section 3.4.6.

Single-blind Placebo Run-in Period

Visit 1 (Day -28 to -14)
Inform the patient about the trial and obtain written informed consent prior to or at Visit 1.
Check eligibility of patient. Patient must be on stable pharmacological treatment of heart failure for at least two weeks prior to Visit 1.
Record personal data, significant medical history/concomitant diseases and concomitant medication/non-drug therapy.
Request the patient to complete the Minnesota Living With Heart Failure Questionnaire (selected countries only).
Perform a physical examination and signs and symptoms review (see Section 3.6.2.).
Perform a standing chest x-ray, unless this has been done within the 6 months prior to Visit 1.
Perform an echocardiogram and measure left ventricular ejection fraction and left ventricular internal diastolic diameter unless this has been performed one week prior to Visit 1.

If no echocardiogram has been performed one week prior to or at Visit 1, arrangements must be made so that results for left ventricular ejection fraction and left ventricular internal diastolic diameter will be available prior to Visit 2 (Randomization).
Perform a 12 lead electrocardiogram at rest.
Withdraw blood for routine laboratory evaluation and request a urine sample for urinalysis with the patient in the fasted state (see Section 3.6.2).
Dispense trial medication and instruct the patient to take one capsule between 6:00 P.M. and 8:00 P.M., every day starting the evening of this visit day, and one capsule between 6:00 A.M. and 8:00 A.M. every day until the next visit. On the day of the next visit, the patient should take the morning dose prior to the visit. Request the patient to return unused medication at the next visit. Instruct the patient to return to the office in 2 to 4 weeks time.

Double Blind Treatment Period

Visit 2 (Day 0, Randomization)
Collect unused trial medication and complete drug log.
Record adverse experiences and concomitant medication.
Request the patient to complete the Minnesota Living With Heart Failure Questionnaire and the EuroQol questionnaire (selected countries only) AFTER recording of adverse experiences and concomitant medication.
In case the patient experienced a morbid event, record occurrence and date and inform the Ciba medical advisor or trial monitor immediately.
Perform a physical examination and signs and symptoms review (see Section 3.6.2.).
Perform a 12 lead electrocardiogram at rest.
Perform pharmacoeconomic assessment.
Withdraw blood after patient has rested 30 minutes in the supine position for neurohormonal measurement.
Withdraw blood for routine laboratory evaluation and request a urine sample for urinalysis with the patient in the fasted state (see Section 3.6.2).
Check eligibility of the patient. If eligible, stratify the patient according to the use of beta blockers and record Randomization Number on the CRF (see Section 3.3).
Dispense trial medication and instruct the patient to take one capsule between 6:00 P.M. and 8:00 P.M. every day, starting the evening of this visit day, and one capsule between 6:00 A.M. and 8:00 A.M. every day until the next visit. On the day of the next visit, the patient should take the morning dose prior to the visit. Request the patient to return unused medication at the next visit. Instruct the patient to return to the office in 2 weeks time.

Visit 3 (Day 14, Week 2)
Collect unused trial medication and complete drug log.
Record adverse experiences and concomitant medication.
Request the patient to complete the EuroQol questionnaire (selected countries only) AFTER recording of adverse experiences and concomitant medication.
In case the patient experienced a morbid event, record occurrence and date and inform the Ciba Medical Advisor or trial monitor immediately.
Perform a physical examination and signs and symptoms review (see Section 3.6.2.).
Perform pharmacoeconomic assessment.
Withdraw blood for routine laboratory evaluation with the patient in the fasted state (see Section 3.6.2).
Check the titration criteria to determine if the dose of trial medication should be increased, decreased or remain unchanged (see Section 3.4.1.).
Dispense trial medication and instruct the patient to take one capsule between 6:00 P.M. and 8:00 P.M. every day, starting the evening of this visit day, and one capsule between 6:00 A.M. and 8:00 A.M. every day until the next visit. On the day of the next visit, the patient should take the morning dose prior to the visit. Request the patient to return unused medication at the next visit. Instruct the patient to return to the office in 2 weeks time.

Visit 4 (Day 28, Week 4)
Collect unused trial medication and complete drug log.
Record adverse experiences and concomitant medication.
Request the patient to complete the Minnesota Living With Heart Failure Questionnaire and the EuroQol questionnaire (selected countries only) AFTER recording of adverse experiences and concomitant medication.
In case the patient experienced a morbid event, record occurrence and date and inform the Ciba Medical Advisor or trial monitor immediately.
Perform a physical examination and signs and symptoms review (see Section 3.6.2.).
Perform pharmacoeconomic assessment.
Withdraw blood for routine laboratory evaluation with the patient in the fasted state (see Section 3.6.2).
Check the titration criteria to determine if the dose of trial medication should be increased, decreased or remain unchanged (see Section 3.4.1.).
Dispense trial medication and instruct the patient to take one capsule between 6:00 P.M. and 8:00 P.M. every day, starting the evening of this visit day, and one capsule between 6:00 A.M. and 8:00 A.M. every day until the next visit. On the day of the next visit, the patient should take the morning dose prior to the visit. Request the patient to return unused medication at the next visit. Instruct the patient to return to the office in 2 weeks time.

Visit 5 (Day 42, Week 6, Month 1.5)
Collect unused trial medication and complete drug log.
Record adverse experiences and concomitant medication.
Request the patient to complete the EuroQol questionnaire (selected countries only) AFTER recording of adverse experiences and concomitant medication.
In case the patient experienced a morbid event, record occurrence and date and inform the Ciba Medical Advisor or trial monitor immediately.
Perform a physical examination and signs and symptoms review (see Section 3.6.2.).
Perform pharmacoeconomic assessment.
Withdraw blood for routine laboratory evaluation with the patient in the fasted state (see Section 3.6.2).
Check the criteria to determine if previous dose level of trial medication should be maintained or decreased (see Section 3.4.1.).
Dispense trial medication and instruct the patient to take one capsule between 6:00 P.M. and 8:00 P.M. every day, starting the evening of this visit day, and one capsule between 6:00 A.M. and 8:00 A.M. every day until the next visit. On the day of the next visit, the patient should take the morning dose prior to the visit. Request the patient to return unused medication at the next visit. Instruct the patient to return to the office in 2 weeks time.

Visit 6 (Day 56, Week 8, Month 2)
Collect unused trial medication and complete drug log.
Record adverse experiences and concomitant medication.
Request the patient to complete the EuroQol questionnaire (selected countries only) AFTER recording of adverse experiences and concomitant medication.
In case the patient experienced a morbid event, record occurrence and date and inform the Ciba Medical Advisor or trial monitor immediately.
Perform a physical examination and signs and symptoms review (see Section 3.6.2.).
Perform pharmacoeconomic assessment.
Dispense trial medication and instruct the patient to take one capsule between 6:00 P.M. and 8:00 P.M. every day, starting the evening of this visit day, and one capsule between 6:00 A.M. and 8:00 A.M. every day until the next visit. On the day of the next visit, the patient should take the morning dose prior to the visit. Request the patient to return unused medication at the next visit. Instruct the patient to return to the office in 9 weeks time.

Visit 7 (Week 17, Month 4)
Collect unused trial medication and complete drug log.
Record adverse experiences and concomitant medication.
Request the patient to complete the Minnesota Living With Heart Failure Questionnaire and the EuroQol questionnaire (selected countries only) AFTER recording of adverse experiences and concomitant medication.
In case the patient experienced a morbid event, record occurrence and date and inform the Ciba Medical Advisor or trial monitor immediately.
Perform a physical examination and signs and symptoms review (see Section 3.6.2.).
Perform an echocardiogram and measure left ventricular ejection fraction and left ventricular internal diastolic diameter.
Perform a 12 lead electrocardiogram at rest.
Perform a pharmacoeconomic assessment.
Withdraw blood after patient has rested 30 minutes in the supine position for neurohormonal measurement.
Withdraw blood for routine laboratory evaluation and request a urine sample for urinalysis with the patient in the fasted state (see Section 3.6.2).
Dispense trial medication and instruct the patient to take one capsule between 6:00 P.M. and 8:00 P.M. every day, starting the evening of this visit day, and one capsule between 6:00 A.M. and 8:00 A.M. every day until the next visit. On the day of the next visit, the patient should take the morning dose prior to the visit. Request the patient to return unused medication at the next visit. Instruct the patient to return to the office in 9 weeks time.

Inform Ciba of the Patient's Designated Dose Level of Trial Medication and Request Re-supply.

Visit 8 (Week 26, Month 6)
Collect unused trial medication and complete drug log.
Record adverse experiences and concomitant medication.
Request the patient to complete the Minnesota Living With Heart Failure Questionnaire and the EuroQol questionnaire (selected countries only) AFTER recording of adverse experiences and concomitant medication.
In case the patient experienced a morbid event, record occurrence and date and inform the Ciba Medical Advisor or trial monitor immediately.
Perform a physical examination and signs and symptoms review (see Section 3.6.2.).
Perform pharmacoeconomic assessment.
Withdraw blood for routine laboratory evaluation and request a urine sample for urinalysis with the patient in the fasted state (see Section 3.6.2).
Dispense trial medication and instruct the patient to take one capsule between 6:00 P.M. and 8:00 P.M. every day, starting the evening of this visit day, and one capsule between 6:00 A.M. and 8:00 A.M. every day until the next visit. On the day of the next visit, the patient should take the morning dose prior to the visit. Request the patient to return unused medication at the next visit. Instruct the patient to return to the office in 13 weeks time.

Visit 9 (Week 39, Month 9)
Collect unused trial medication and complete drug log.
Record adverse experiences and concomitant medication.
Request the patient to complete the Minnesota Living With Heart Failure Questionnaire and the EuroQol questionnaire (selected countries only) AFTER recording of adverse experiences and concomitant medication.
In case the patient experienced a morbid event, record occurrence and date and inform the Ciba Medical Advisor or trial monitor immediately.
Perform a physical examination and signs and symptoms review (see Section 3.6.2.).
Perform pharmacoeconomic assessment.
Dispense trial medication and instruct the patient to take one capsule between 6:00 P.M. and 8:00 P.M. every day, starting the evening of this visit day, and one capsule between 6:00 A.M. and 8:00 A.M. every day until the next visit. On the day of the next visit, the patient should take the morning dose prior to the visit. Request the patient to return unused medication at the next visit. Instruct the patient to return to the office in 13 weeks time.

Visit 10 (Week 52, Month 12, Year 1)
Collect unused trial medication and complete drug log.
Record adverse experiences and concomitant medication.
Request the patient to complete the Minnesota Living With Heart Failure Questionnaire and the EuroQol questionnaire (selected countries only) AFTER recording of adverse experiences and concomitant medication.
In case the patient experienced a morbid event, record occurrence and date and inform the Ciba Medical Advisor or trial monitor immediately.
Perform a physical examination and signs and symptoms review (see Section 3.6.2.).
Perform an echocardiogram and measure left ventricular ejection fraction and left ventricular internal diastolic diameter.
Perform a 12 lead electrocardiogram at rest.
Perform a standing chest x-ray unless this has been done within the preceding 2 months.
Perform pharmacoeconomic assessment.
Withdraw blood after patient has rested 30 minutes in the supine position for neurohormonal measurement.
Withdraw blood for routine laboratory evaluation and request a urine sample for urinalysis with the patient in the fasted state (see Section 3.6.2).
Dispense trial medication and instruct the patient to take one capsule between 6:00 P.M. and 8:00 P.M. every day, starting the evening of this visit day, and one capsule between 6:00 A.M. and 8:00 A.M. every day until the next visit. On the day of the next visit, the patient should take the morning dose prior to the visit. Request the patient to return unused medication at the next visit. Instruct the patient to return to the office in 13 weeks time.

Visit 11 (Week 65, Month 15)

Same visit procedures as for Visit 9. In addition:
Inform Ciba of the patient's designated dose level of trial medication and request re-supply.

Visit 12 (Week 78, Month 18, Year 1.5)

Same visit procedures as for Visit 9. In addition:
Perform an echocardiogram and measure left ventricular ejection fraction and left ventricular internal diastolic diameter.
Withdraw blood for routine laboratory evaluation and request a urine sample for urinalysis with the patient in the fasted state (see Section 3.6.2).

Visit 13 (Week 91, Month 21)

Same visit procedures as for Visit 9.

Visit 14 (Week 104, Month 24, Year 2)

Same visit procedures as for Visit 10.

Visit 15 (Week 117, Month 27)

Same visit procedures as for Visit 9. In addition:
Inform Ciba of the patient's designated dose level of trial medication and request re-supply.

Visit 16 (Week 130, Month 30, Year 2.5)

Same visit procedures as for Visit 9. In addition:
Perform an echocardiogram and measure left ventricular ejection fraction and left ventricular internal diastolic diameter.
Withdraw blood for routine laboratory evaluation and request a urine sample for urinalysis with the patient in the fasted state (see Section 3.6.2).

Visit 17 (Week 143, Month 33)

Same visit procedures as for Visit 9.

Visit 18 (Week 156, Month 36, Year 3)

Same visit procedures as for Visit 10.

Visit 19 (Week 169, Month 39)

Same visit procedures as for Visit 9. In addition:
Inform Ciba of the patient's designated dose level of trial medication and request re-supply.

Visit 20 (Week 182, Month 42, Year 3.5)

Same visit procedures as for Visit 9. In addition:
Perform an echocardiogram and measure left ventricular ejection fraction and left ventricular internal diastolic diameter.
Withdraw blood for routine laboratory evaluation and request a urine sample for urinalysis with the patient in the fasted state (see Section 3.6.2).

Visit 21 (Week 195, Month 45)

Same visit procedures as for Visit 9.

Visit 22 (Week 208, Month 48, Year 4)

Same visit procedures as for Visit 10.

Final Visit:

Same visit procedures as for Visit 10.
No further trial medication will be dispensed to the patient.
Complete the Termination Sheet of the CRF and attach it to this visit report.

3.6.2. Procedure Descriptions

Adverse Experiences

At each visit during the trial, except Visit 1, all new or continuing adverse experiences which were not present at the initial visit (Visit 1) must be recorded. A detailed instruction on how adverse experiences will be recorded during the trial is given in Section 3.8

Physical Examination and Signs and Symptoms Review

Blood Pressure

Systolic and diastolic blood pressure will be measured at each visit according to the WHO guidelines with a mercury sphygmomanometer (two measurements in the sitting position after 5 minutes resting followed by one measurement in the standing position after at least 2 minutes of equilibration). Blood pressure will be measured by the same clinician using the same sphygmomanometer on the same patient on the dominant arm. All measurements are to the nearest 2 mm Hg. For determination of diastolic blood pressure, phase V (disappearance of the Korotkoff sound) will be used. The measurements will always be carried out at the same time of the day after intake of the morning dose.

Pulse Rate

Pulse rate will be measured at each visit once in the sitting position after 5 minutes resting followed by one measurement in the standing position after at least 2 minutes of equilibration. Pulse rate will be measured for 30 seconds and before blood pressure measurements.

Weight

Body weight will be measured at each visit using the same scale.

Signs and symptoms review
Signs and symptoms of CHF will be reviewed by the physician at each
visit. Signs' and symptoms' scores are as follows:

| Paroxysmal | | |
|---|---|---|
| nocturnal dyspnea | Fatigue | Edema |
| 0 (absent) | 0 (absent) | 0 (absent) |
| 1 (present) | 1-4 (present) | 1 (trace) |
| | | 2 (feet and ankles) |
| | | 3 (lower legs or thighs) |
| | | 4 (sacrum) |

| Dyspnea | | |
|---|---|---|
| at rest | Orthopnea | Rales |
| 0 (absent) | 0 (absent) | 0 (absent) |
| 1 (present) | 1 (lying) | 1 (basilar only) |
| | 2 (0°-45°) | 2 (>⅓ of lung fields) |
| | 3 (46°-90°) | |

| | Jugular venous | |
|---|---|---|
| Dyspnea on effort | distention −45° | Third heart sound |
| 0 (absent) | 0 (absent) | 0 (absent) |
| 1-4 (present) | 1 (present) | 1 (present) |

NYHA class will be scored as 1 (Class I), 2 (Class II), 3 (Class III), and 4 (Class IV) (Ref. 11).

Chest X-ray

A posterior/anterior and lateral chest x-ray will be performed at Visit 1 (unless one has been performed in the 6 months prior to Visit 1) and every 12 months thereafter (unless one has been performed 2 months prior).

Left Ventricular Ejection Fraction/Left Ventricular Internal Diastolic Diameter

Left ventricular ejection fraction and left ventricular internal diastolic diameter using echocardiography will be determined at Visit 1 or prior to Visit 2 (result must be available at Visit 2), and Visits 7, 10 and every 6 months thereafter.

ECG

A 12-lead electrocardiogram at rest will be performed at Visits 1, 2, 7, 10 and every 12 months thereafter.

Congestive Heart Failure Quality of Life Assessment

Quality of life will be assessed at Visits 1, 2, 4, 7, 8, 9, 10 and every 3 months thereafter using the Minnesota Living with Heart Failure Questionnaire (Ref. 12) in selected countries. The questionnaire will be completed by the patient in a quiet room after collection of adverse experience and concomitant medication information but before any other trial procedures are performed.

Routine Laboratory Evaluations

Routine laboratory evaluations include hematology, blood chemistry, and urinalysis and will be performed at Visits 1, 2, 7, 8, and every 6 months thereafter. At Visits 3, 4, and 5, serum creatinine, BUN, sodium, potassium, chloride and bicarbonate will be measured.

Hematology: RBC, hemoglobin, hematocrit, WBC, differential count, platelets.

Blood chemistry: glucose, creatinine, uric acid, BUN, potassium, sodium, chloride, calcium, phosphate, bicarbonate, total protein, albumin, SGOT (AST), SGPT (ALT), alkaline phosphatase, total bilirubin, total cholesterol.

Urinalysis: protein, glucose, blood.

All patients with laboratory tests containing clinically significant abnormal values will be followed regularly until the values return to normal ranges or until a valid reason, other than drug-related adverse experience, is identified.

Female patients of childbearing potential will have a serum pregnancy test at every visit

Neurohormonal Measurements

Neurohormonal measurements will be performed in all patients at Visit 2 after 30 minutes in the supine position and include measurement of plasma renin activity (PRA), aldosterone, norepinephrine, endothelin and brain natriuretic peptide. At Visits 7, 10 and every 12 months thereafter norepinephrine will be measured in the supine position in all patients.

Pharmacoeconomic Evaluation

A pharmacoeconomic assessment, including the EUROQOL patient questionnaire (Ref. 13) in selected countries, will be performed at Visit 2 and all subsequent visits.

3.7. Removal of Patients from Trial or Analysis

Removal of Patients from Trial Treatment

All randomized patients (including patients with morbid events other than death) should remain on trial treatment until death or until trial end, except in case of the following events, which are considered sufficient reason for a patient to discontinue trial treatment:
whenever the patient decides that it is in his/her best interest
whenever the investigator considers it advisable or in the patient's best interest
intolerable adverse experiences
presence of life-threatening laboratory abnormality despite manipulation of trial therapy and/or background treatment
positive pregnancy-test results in a patient who decides to carry pregnancy to term.

Patients should be removed from trial treatment if, after alteration of the dose level of trial treatment and background treatment, the following criteria are met:
persistent standing systolic blood pressure<80 mm Hg

OR
symptoms of hypotension (syncope, faintness, orthostatic dizziness)

Patients still alive at the time of premature discontinuation from double-blind trial treatment should continue to visit the investigator according to the protocol until trial end. In these cases, no trial medication will be dispensed to the patient and data collection will be made including: date of visit, date of occurrence of morbid event(s), and cardiovascular-related concomitant medications. The date and principal reason for the patient's permanent discontinuation from double-blind trial medication must be collected and entered on the appropriate CRFs.

Patients still alive at the time of premature discontinuation from double-blind trial treatment may not enroll in any subsequent investigational trials until trial end.

Removal of Patients from the Trial

All efforts should be made to keep patients in the trial, including patients who have permanently discontinued trial medication (as described above). Patients will be considered discontinued from the trial only if they undergo heart transplantation or are lost to follow-up after exhausting all means of contact.

A genuine effort must be made to determine the reason(s) why a patient fails to return for the necessary visits or is discontinued from the trial. This information should be recorded on the appropriate case report forms and on the Termination sheet.

If a Patient Discontinues from the Trial with Reasons other than Heart Transplantation, All Efforts Should be Made to Collect the Information Related to the Patient's Death Needed for the Termination Sheet.

It is agreed that, for reasonable cause, either the investigator or the sponsor, Ciba, may terminate this trial, provided a written notice is submitted at a reasonable time in advance of intended termination.

Removal of Patients from Analysis

There are no pre-planned reasons for removal of patients from the all-randomized-patients (intention-to-treat) analysis.

3.8. Recording Adverse Experiences

At each visit during the trial, after Visit 1, all new or continuing adverse experiences which were not present at the initial visit (Visit 1) must be recorded. Any medical condition present at the initial visit, which remains unchanged or improves, should not be recorded as an adverse experience at subsequent visits. However, if there is deterioration of a medical condition that was present at the initial visit, then this should also be considered a new adverse experience and reported. This information is obtained by questioning and/or examining the patient.

Trial drug relationship for each adverse experience should be determined by the investigator using the following explanations:

Not Related
The experience is clearly related to other factors such as the patient's clinical state, therapeutic interventions, or concomitant drugs administered to the patient.

Unlikely
The experience was most likely produced by other factors such as the patient's clinical state, therapeutic interventions, or concomitant drugs administered to the patient,
and does not follow a known response pattern to the trial drug.

Possible
The experience follows a reasonable temporal sequence from the time of drug administration,
and/or follows a known response pattern to the trial drug,
but could have been produced by other factors such as the patient's clinical state, therapeutic interventions, or concomitant drugs administered to the patient.

Probable
The experience follows a reasonable temporal sequence from the time of drug administration,
and follows a known response pattern to the trial drug,
and cannot be reasonably explained by other factors such as the patient's clinical state, therapeutic interventions, or concomitant drugs administered to the patient.

Highly Probable
The experience follows a reasonable temporal sequence from the time of drug administration,
and follows a known response pattern to the trial drug,
and cannot be reasonably explained by other factors such as the patient's clinical state, therapeutic interventions, or concomitant drugs administered to the patient,
and either occurs immediately following trial drug administration, or improves on stopping the drug, or reappears on repeat exposure, or there is a positive reaction at the application site.

Note: The decision to perform a formal challenge must be made by the Ciba Clinical Research Physician and the investigator after reviewing the patient's complete history.

Trial drug(s) includes the drug(s) under evaluation, the reference drug(s), placebo, or any other drug(s) required by the protocol.

Severity of an adverse experience is defined as a qualitative assessment of the degree of intensity of an adverse experience as is determined by the investigator or reported to him/her by the patient. The assessment of severity is made irrespective of drug relationship or seriousness of the experience and should be evaluated according to the following scale:

1=Mild

2=Moderate

3=Severe

Any serious adverse experience (SAE), including a serious clinical laboratory abnormality, must be reported to Ciba within 24 hours of learning that the SAE occurred.

The definition of a serious adverse experience (SAE) is as follows:

A serious adverse experience is considered to be any experience that suggests a significant hazard, contraindication, side effect, or precaution. In that regard, medical judgment is required in the evaluation of incoming information. As a rule, a serious adverse experience includes any experience that is fatal or life-threatening, is permanently disabling, requires inpatient or prolonged hospitalization, or is a congenital anomaly, cancer, or a drug overdose.

The patient must be followed carefully until the condition disappears and/or the etiology is identified.

Although the present invention has been described in considerable detail with reference to certain preferred versions thereof, other versions are possible without departing from the spirit and scope of the preferred versions contained herein.

What is claimed is:

1. A pharmaceutical composition comprising valsartan, alone or in combination with at least another therapeutic agent in the presence of a pharmaceutically acceptable carrier for the prevention or treatment of Atrial fibrillation (AF) in patients having heart failure.

2. Use of a pharmaceutical composition comprising valsartan, alone or in combination with at least another therapeutic agent in the presence of a pharmaceutically acceptable carrier for the preparation of a medicament for the prevention or treatment of AF in patients having heart failure.

3. A method for preventing or reducing the incidence of AF in patients with heart failure comprising administering a therapeutically effective amount of a pharmaceutical composition comprising valsartan, alone or in combination with at least another therapeutic agent in the presence of a pharmaceutically acceptable carrier.

4. The method of claim 3, wherein valsartan is administered in a daily dose of from about 80 mg to about 320 mg.

5. A method of reducing morbidity or mortality in a patient having heart failure comprising administering a therapeutically effective amount of valsartan, alone or in combination with at least another therapeutic agent in the presence of a pharmaceutically acceptable carrier.

6. Pharmaceutical composition, use, method according to any of the preceding claims wherein the therapeutic agent is selected from the group consisting of anti-hypertensive agents, anti-obesity agents, anti-diabetic agents, beta-blockers, inotropic agents and hypolipidemic agents.

7. Pharmaceutical composition, use, method according to claim 6, wherein the anti-hypertensive agent is an ACE inhibitor.

8. Pharmaceutical composition, use, method according to claim 7, wherein valsartan is combined with an ACE inhibitor and a beta-blocker.
